(19) United States
(12) Patent Application Publication
(10) Pub. No.: US 2006/0206151 A1
(43) Pub. Date: Sep. 14, 2006

(54) Heart rhythm management system

(76) Inventor: Fei Lu, Maple Grove, MN (US)

Correspondence Address: FISH & RICHARDSON P.C., PO BOX 1022, MINNEAPOLIS, MN, 55440-1022, US

(21) Appl. No.: 11/355,831

(22) Filed: Feb. 16, 2006

Related U.S. Application Data
(60) Provisional application No. 60/653,350, filed on Feb. 16, 2005.

(51) Int. Cl. A61N 1/39; A61N 1/362

(52) U.S. Cl. 607/4

(57) ABSTRACT

The invention provides for a heart rhythm management system that includes a battery module and either or both a pacing module and a defibrillator module. The modules have connection ports for releasably connecting one module to another. The invention also provides methods of using such a heart rhythm management system.

### CROSS-REFERENCE TO RELATED APPLICATIONS

This application claims the benefit of priority under 35 U.S.C. §119(e) of U.S. Application No. 60/653,350 filed Feb. 16, 2005. The disclosure of the prior application is incorporated by reference in its entirety.

### TECHNICAL FIELD

This invention relates to a hearth rhythm management system, and more particularly to pacing modules, defibrillator modules, and battery modules that can be releasably connected to one another.

### BACKGROUND

A heart has a natural pacemaker in the upper right atrium called the sinoatrial (SA) node that produces electrical signals. These signals travel down through the conduction pathways and cause the heart's chambers to contract. The right and left atria contract before the right and left ventricles. The electrical impulse travels from the SA node to the atrioventricular (AV) node, then continues down the conduction pathways via the His bundle. The His bundle divides into right and left pathways (the Purkinje fibers) to provide electrical stimulation to both ventricles. Normally, the electrical impulse moves through the heart and the heart contracts about 60 to 100 times a minute. For a variety of reasons, the heart's electrical system can malfunction and cause arrhythmia (i.e., fast, slow, and/or irregular heart rhythms). Certain types of arrhythmia can be life-threatening and can necessitate anti-arrhythmic therapy using drugs and/or an implantable cardiac device.

### SUMMARY

The invention provides for a heart rhythm management system that includes a battery module and either or both a pacing module and a defibrillator module. The modules have connection ports for releasably connecting one module to another. The invention also provides for methods of using heart rhythm management systems of the invention.

In one aspect, the invention provides a heart rhythm management system. Such a heart rhythm management system includes a battery module, and either or both a pacing module or a defibrillator module. The battery module, the pacing module, and the defibrillator module each have at least one connection port adapted for releasably connecting the module to another module.

Certain embodiments of a heart rhythm management system include a battery module and a pacing module; certain embodiments include a battery module and a defibrillator module; and certain embodiments include a battery module, a pacing module, and a defibrillator module. In certain embodiments, the pacing module and/or the defibrillator module lacks an internal battery component.

A heart rhythm management system of the invention generally includes a capacitor module. A separate capacitor module can include at least a first and a second connection port. Generally, the first connection port is adapted for releasably connecting the capacitor module to a battery module, and the second connection port is adapted for releasably connecting the capacitor module to the pacing module and/or the defibrillator module. Alternatively, a battery module can further include a capacitor module.

Typically, modules are connected to one another with connectors. Representative connectors include electrical wires and fiber optics. In certain embodiments, the connectors also can have electrodes for sensing, pacing, and/or delivering high energy shocks. Connectors can be connected to a connection port on a module using, for example, a snap-fit connection, a clamp connection, a male-female connection, or a surface connection. In certain embodiments, a connector can be secured to one of the connection ports described above using screws. In other embodiments, a connection port can include one or more screws.

In another aspect, the invention provides methods of managing an patient's heart rhythm. Such a method can include implanting a battery module and a pacing module in the patient, and releasably connecting the battery module and the pacing module; implanting a battery module and a defibrillator module in the patient, and releasably connecting the battery module and the defibrillator module; implanting a battery module, a pacing module, and a defibrillator module in the patient, and releasably connecting the battery module, the pacing module, and the defibrillator module; implanting a defibrillator module, and releasably connecting a defibrillator module to an existing system, wherein the existing system comprises a pacing module and a battery module; or replacing a battery module in an patient's existing heart rhythm management system with a replacement battery module, and releasably connecting the replacement battery module to the existing system. The patient's heart rhythm is managed by the implanted modules.

In some embodiments of a heart rhythm management system of the invention, the battery module can be remotely positioned from either or both the pacing module and the defibrillator module when implanted in a patient. For example, a battery module can be implanted in the patient's abdomen.

In still another aspect, the invention provides an article of manufacture. An article of manufacture of the invention can include a battery module, connectors, and either or both a pacing module and a defibrillator module. An article of manufacture of the invention can further include a capacitor module. An article of manufacture of the invention can include a battery module, and connectors. An article of manufacture of the invention can include a pacing module, and connectors. An article of manufacture of the invention can include a defibrillator module, and connectors. Generally, the connectors are electrical wires or fiber optics. In certain embodiments, the connectors include electrodes for sensing, pacing, and/or delivering high energy shocks.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIGS. 1A-1F are schematics of a patient implanted with various embodiments of a heart rhythm management system of the invention.

Like reference symbols in the various drawings indicate like elements.

### DETAILED DESCRIPTION

Over the past twenty years, implantable cardiac devices have evolved from relatively bulky, crude, and short-lived devices that provide simple energy therapy to complex, long-lived, and miniaturized devices that can provide a wide variety of pacing, cardioversion, and/or defibrillation therapies. Numerous other functions have been incorporated into implantable cardiac devices including, for example, an enhanced capacity to detect and discriminate cardiac arrhythmias, provision of staged therapies appropriate to the detected arrhythmia, and data storage and uplink telemetry of data related to arrhythmia episodes and applied therapies.

Throughout the course of these improvements, each generation of implantable cardiac devices includes common components located within a housing. Minimally, these components include one or more batteries, electronic circuitry for detecting arrhythmias, and electronic circuitry for moving energy out through the leads. The housing is typically formed of a biologically-compatible material such as titanium and usually is hermetically sealed to be impervious to body fluids.

At the same time, numerous improvements have been made in leads and electrodes that have enabled the energy to be precisely delivered to particular regions of the heart. Such focused energy delivery reduces the amount of energy required to pace or shock the heart, as well as reduces or eliminates the risk of delivering energy to a region of the heart that doesn't need it. Moreover, the output circuitry has been improved to provide monophasic, biphasic, or multi-phase pulse or shock waveforms, sometimes with particular combinations of electrodes, that also lowers the energy required to alleviate arrhythmias.

Nerve-, brain-, muscle-, and other organ-stimulating implantable devices are known in the art for treating a variety of conditions. The present invention will be described in relation to a heart rhythm management system, but it is not intended that the invention be limited to cardiac devices when it can be advantageously implemented in other tissues and organs.

Heart Rhythm Management System

The present invention describes a heart rhythm management system in which the components of conventional implantable cardiac devices have been modularized. A heart rhythm management system of the invention includes at least a battery module and further includes either or both a pacing module and a defibrillator module. The heart rhythm management system described herein provides the physician with much more flexibility than do conventional implantable devices. For example, the battery modules, pacing modules and defibrillator modules are smaller than conventional devices containing a multitude of functions within a single housing. In addition, the modules can be remotely positioned from one another. For example, the battery module can be positioned in the abdomen while the pacing and/or defibrillator modules are positioned in the chest wall. These features of a heart rhythm management system reduce or eliminate the outward visibility that is so problematic to patients implanted with a much larger conventional device.

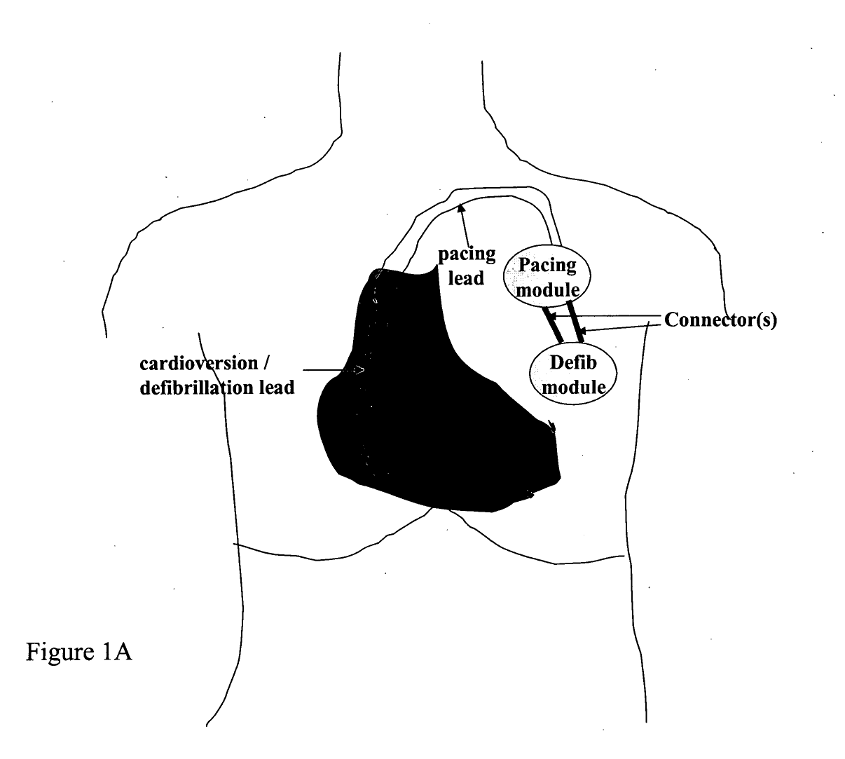

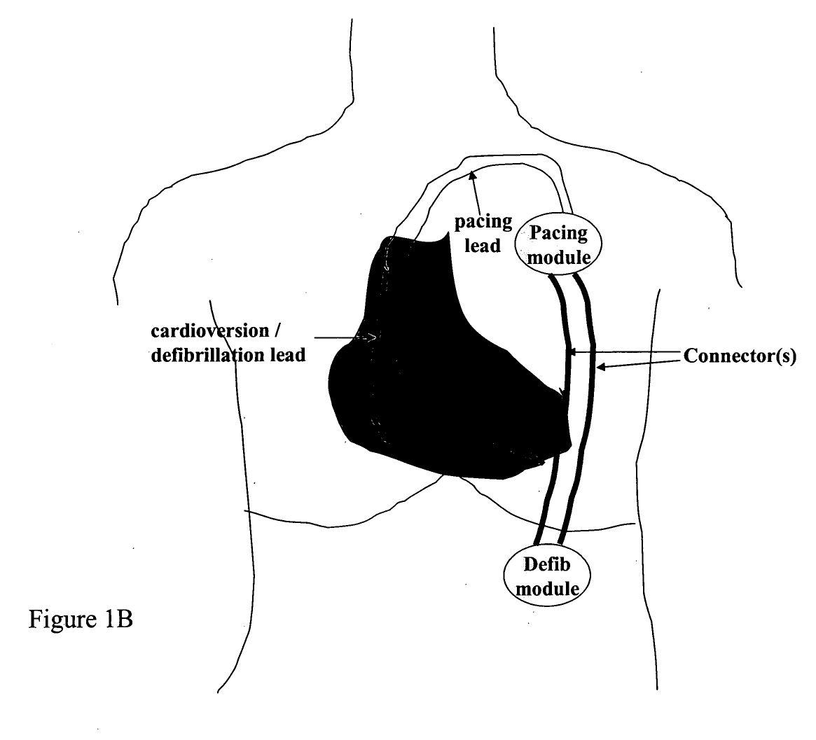

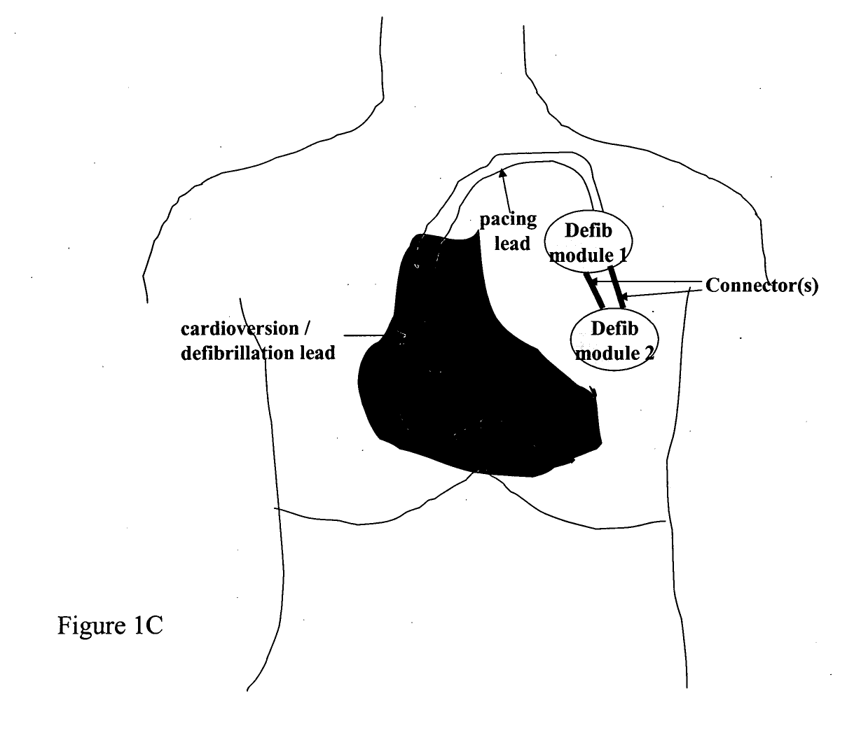

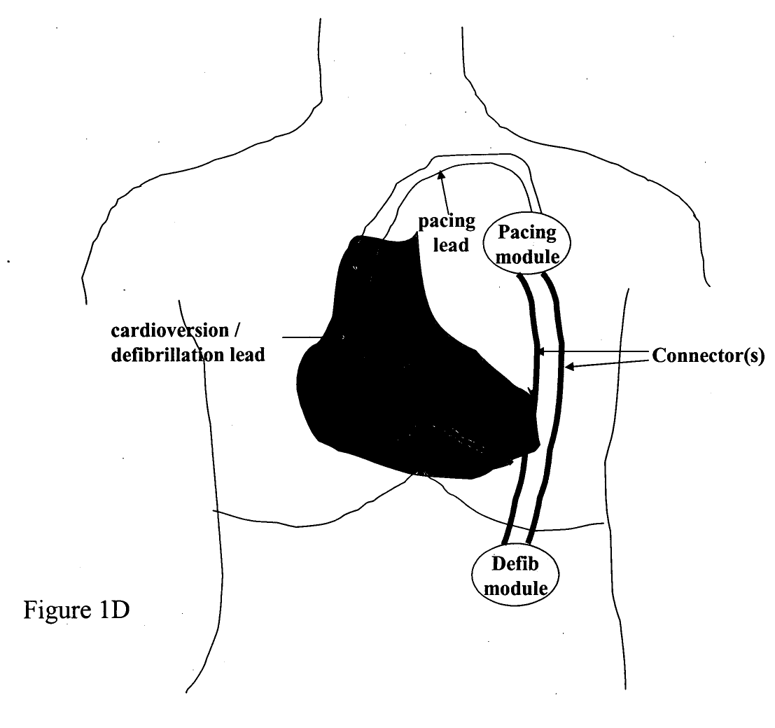

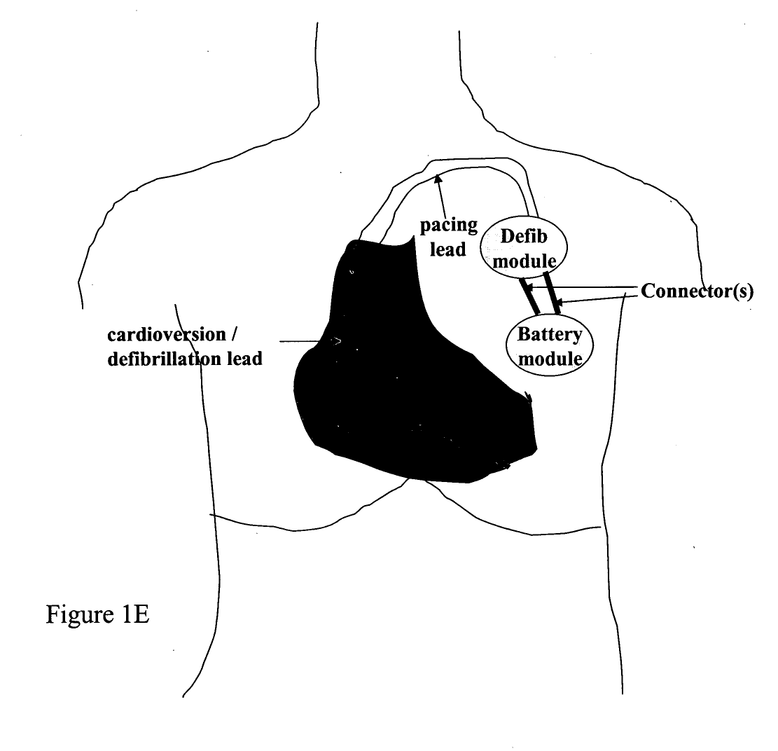

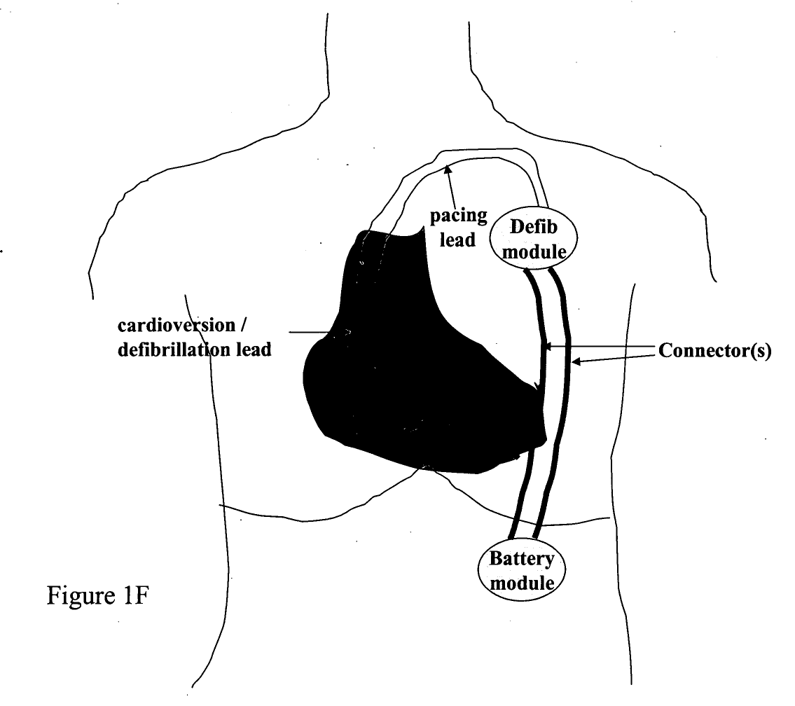

In various embodiments, a heart rhythm management system of the invention can include a battery module and a pacing module; a battery module and a defibrillator module; or a battery module, a pacing module, and a defibrillator module. If necessary, multiple modules such as multiple defibrillator modules can be used in a heart rhythm management system of the invention. In addition, an existing heart rhythm management system already implanted in a patient can be upgraded by adding a module (e.g., a defibrillator module), and modules (e.g., a battery module) can be replaced without replacing the entire system. The particular combination of modules utilized in a patient's heart rhythm management system is determined primarily by the patient's medical condition. A number of non-limiting examples of heart rhythm management systems of the invention are shown in FIGS. 1A-1F.

Battery Modules

A heart rhythm management system described herein includes at least one battery module. A battery module as used herein has a housing that contains at least one battery component. A battery component can be either a low-voltage or a high-voltage battery component. The battery component(s) used in a battery module can be lithium-iodide battery components, or any other type of energy-producing battery known in the art. Over the last several years, the energy requirements for powering both low-voltage sensing and pacing and high-voltage shocks have been reduced, while the energy density of a battery has been increased. A battery module as described herein also has at least one connection port for releasably connecting the battery module to another module.

Capacitors are pairs of positive and negative plates that store an electrical charge or energy. The plates in a pair are positioned very close to one another with an insulator in between to prevent the plates from touching one another. Capacitors can be made using a number of different materials including aluminum, polypropylene, polyester, polystyrene, and/or polycarbonate, to name a few.

In one embodiment, a capacitor component can be integrated into a battery module. Alternatively or additionally, a heart rhythm management system of the invention can include a separate capacitor module in addition to a battery module. A capacitor module that is separate from a battery module has a housing that contains at least one capacitor component. A capacitor module has at least two connection ports for releasably connecting the capacitor module to a battery module and either a pacing module or a defibrillator module.

Various batteries and capacitors or improvements thereof are described in U.S. Pat. Nos. 5,370,663; 5,370,669; 5,405,363; 5,527,346; 5,749,911; and 5,827,326, and can be incorporated into a battery module and/or a capacitor module.

Pacing Modules

Pacing modules continuously monitor the heart rate and generate an electrical impulse to pace the heart if the heart rate becomes too slow. As used herein, a pacing module includes a housing, electronic detection and pacing circuitry, and pacing leads bearing at least sensing and pacing electrodes. A pacing module also has at least one connection port for releasably connecting the pacing module to a battery module, or to a capacitor module and a battery module. A pacing module also can have connection ports for releasably connecting another pacing module or a defibrillator module. Pacing modules as used herein may or may not contain an internal battery component. In certain embodiments, the pacing module lacks an internal battery component and relies entirely on a battery module. In other embodiments, the pacing module may have an internal battery component (e.g., a back-up and/or a supplemental battery) but still can be connected to a battery module.

Pacing leads generally are insulated flexible wires that transmit electrical signals from the pacing module to the heart. Pacing leads also may relay signals from the heart to the pacing module. The proximal end of a pacing lead is attached to the pacing module, and the distal end of a pacing lead is positioned in the atrium or in the ventricle of a patient's heart. A description of leads can be found, for example, in U.S. Pat. No. 4,932,407.

Any pacing circuitry known in the art can be used in a pacing module described herein. Pacing circuitry generally includes a pulse generator for generating cardiac pacing pulses. A pulse generator may include timing circuitry for defining ventricular pacing intervals, refractory intervals, and blanking intervals under control of a microprocessor. Pulse generators can be set to a fixed-rate (asynchronous) or demand (synchronous) mode as is known in the art. Signals triggering generation of pacing pulses and signals indicative of the occurrence of R-waves are communicated to the pacing circuitry using a bidirectional data bus. Single-chamber or dual-chamber pacing and sensing functions that employ suitable pacing circuitry and suitable unipolar or bipolar electrodes are known in the art and can be included in a pacing module described herein.

The North American Society of Pacing and Electrophysiology (NASPE) and the British Pacing and Electrophysiology Group (BPEG) have developed a code to describe various pacing modes. See, for example, Bernstein, 1991, Tex. Heart Inst. J, 18(4):299-300 and Bernstein et al., 2002, PACE, 25:260-4. Similar to conventional pacemakers, the programming of pacing modules can be performed non-invasively by an electrophysiologist or a cardiologist.

Defibrillator Modules

Defibrillator modules continuously monitor the heart rate and are able to correctly identify normal rhythms as well as abnormal rhythm disturbances. If the heart rate is dangerously fast, a defibrillator module can deliver bursts of fast stimuli to terminate the arrhythmia (antitachycardia pacing) or can deliver a small electrical shock to convert the arrhythmia to a normal rhythm (cardioversion and defibrillation). As used herein, a defibrillator module includes a housing, electronic circuitry for pacing, cardioversion, and/or defibrillation, and at least one shocking lead. Defibrillator modules also have at least one connection port for releasably connecting the defibrillator module to a battery module, or to a capacitor module and a battery module. Defibrillator modules as used herein may or may not contain an internal battery component. In certain embodiments, the defibrillator module lacks an internal battery component and relies entirely on a battery module. In other embodiments, the defibrillator module may have an internal battery component (e.g., a back-up and/or a supplemental battery) but still can be connected to a battery module.

Any cardioversion/defibrillation circuitry known in the art can be used in a defibrillator module described herein. A defibrillator module can be controlled by the operation of a microprocessor using technology known in the art. A microprocessor can be linked to cardioversion/defibrillation circuitry using a bi-directional data/control bus, and also can have interrupt and signal lines to control operation of the high-voltage charging and output circuitry.

Cardioversion/defibrillation leads carry electrical impulses from the defibrillator module to the heart. Cardioversion/defibrillation leads also can send information from the heart back to the defibrillator module for monitoring the heart. Cardioversion/defibrillation leads typically are flexible insulated wires with an electrode at the distal end for sending and pacing and/or one or more shocking coils that generally have a large surface area for delivering electrical shocks. A description of leads can be found, for example, in U.S. Pat. No. 4,932,407.

Connections Between Modules

The modules of a heart rhythm management system of the invention are connected to each other with connectors. The connectors can be any material that allows for transfer of signals, data, and/or energy from one module to another. For example, connectors can be electrical wires or fiber optics. In some embodiments, the connectors themselves can include electrodes on their surfaces for sensing and/or delivering energy at locations between modules. The manner in which modules are connected (e.g., in series and/or in parallel) is dependent upon the particular combination(s) of modules and can be determined as clinically indicated by those skilled in the art.

Each module suitable for use in a heart rhythm management system of the invention includes at least one connection port for connecting the module to another module via connectors. One portion of the connection port, the “module portion,” is in communication with the circuitry inside the module. Another portion of the connection port, the “connector portion,” can be any configuration that allows for a releasable connection between a module and one or more connectors. Connection ports can include, but are not limited to, snap-fit connections, clamp connections, male-female connections, and/or surface connections. A connector can be secured to one of the connection ports described above using screws. Alternatively, a connection port can include one or more screws.

A heart rhythm management system of the invention may use a single type of connection port or more than one type of connection port. For example, pacing modules and defibrillator modules may have one type of connection port for releasably connecting to a battery module, while battery modules may have a different type of connection port for releasably connecting to a pacing module or defibrillation modules. In addition, a battery module may have different types of connection ports for connecting a pacing module and a defibrillator module. In some embodiments, the connection ports and the connectors, or the ends of the connectors, can be color-coded or otherwise labeled for connection to the appropriate module.

In certain embodiments, the pacing module and the defibrillator module can contain software and/or hardware to interface wirelessly between the two modules. In order to deliver energy, however, the connection between the pacing module or the defibrillator module and the battery module likely is not wireless. In a wireless embodiment, the components for interfacing between modules (e.g., software and/or hardware) may be considered to be connectors and connection ports within the scope of these embodiments.

The electrical leads that deliver energy from a pacing module or a defibrillator module to the target tissue also can be releasably connected to the respective module using connection ports such as those described herein or any other releasable connection known in the art. Releasable connection of the leads allows for replacement of the pacing or defibrillator module without replacing the leads.

Methods of Using a Heart Rhythm Management System

Implantation of the modules of a heart rhythm management system usually is an outpatient procedure done in a cardiac catheterization laboratory, an electrophysiology laboratory, or an operating room, and generally takes a few hours. A local anesthetic usually is applied to the skin. An incision is made under the collarbone, and the appropriate modules are implanted under the skin. The pacing module and the defibrillator module usually are implanted in the chest underneath the skin near the collarbone, while the battery module can be implanted near the pacing and/or defibrillator module (e.g., in the chest wall, or near the collarbone), or can be remotely positioned relative to the pacing and/or defibrillator modules (e.g., in the abdomen).

To position the leads, the distal end of a lead is inserted through a vein near the pacing and/or defibrillator module (e.g., the subclavian, axillary, or cephalic vein) into the heart's chambers. The tip of the lead usually is implanted onto the heart's inner wall. Sometimes it is necessary to position multiple leads (e.g., in both the atrium and the ventricle). Alternatively, a single lead can be used to sense and deliver energy to both chambers. The number and precise position of leads depends on the patient's particular needs.

After the distal ends of the leads are positioned, the leads are tested to make sure they are correctly positioned and are functioning correctly, and the leads are then fixed to underlying tissues to prevent movement. The proximal ends of the leads are connected to the pacing module and/or defibrillator module. The physician often will deliberately induce arrhythmia to confirm that the modules are functioning properly.

The modules described herein can be implanted in a patient in any number of combinations, depending on the patient's need for heart rhythm management. Without limitation, a battery module and a pacing module can be implanted in a patient and releasably connected to one another; a battery module and a defibrillator module can be implanted in a patient and releasably connected to one another; or a battery module, a pacing module, and a defibrillator module can be implanted in a patient and releasably connected to one another. As discussed above, a capacitor component can be included as part of a battery module, or can be included as a separate capacitor module in a heart rhythm management system of the invention.

The heart rhythm management system allows for an additional module to be implanted and releasably connected to other modules that had been previously implanted. In another embodiment, one module in an patient's existing heart rhythm management system can be replaced with a replacement module and releasably connected to the existing system. As used herein, a replacement module can be identical to the original module implanted, or the replacement module can be, for example, updated to include current technology. The releasable connections allow for replacement of only the expired or malfunctioning module itself without removal and disposal of the entire system.

Pacing modules generally are indicated for treatment of slow heart rates that may result from heart block or sinus node dysfunction. Specifically, indications for pacing module implantation include, but are not limited to, sick sinus syndrome, symptomatic sinus bradycardia, brady-tachy syndrome, atrial fibrillation with a slow ventricular response, atrioventricular block, chronotropic incompetence, long QT syndrome, cardiomyopathy (hypertrophic or dilated), and/or refractory neurocardiogenic syncope. Defibrillator modules generally are indicated for treatment of dangerous arrhythmias such as certain types of ventricular tachycardia and ventricular fibrillation.

Articles of Manufacture

The invention also provides for articles of manufacture containing various combinations of the modules disclosed herein, and also may contain means for connecting the modules (e.g., connecting wires). For example, an article of manufacture can include a battery module as discussed above, connectors as discussed above, and either or both a pacing module or a defibrillator module as discussed above. Such an article of manufacture of the invention also can include a capacitor module as discussed above. In various embodiments, articles of manufacture of the invention can include a battery module and connectors; a pacing module and connectors; or a defibrillator module and connectors.

Articles of manufacture of the invention further can include instructions for implanting the particular module or modules, and further may contain information for releasably connecting one module to another. The instructions in an article of manufacture of the invention also can include programming information for either or both a pacing module or a defibrillator module. An article of manufacture of the invention also can include, for example, a card for the patient to carry. Such a card can identify the particular module(s) implanted in the patient, and also can contain information about the programming of the modules or any other relevant patient and/or physician information.

In accordance with the present invention, there may be employed conventional cardiology techniques that are within the skill of the art. Such techniques can be performed using computer generated models, an in vitro system, in vivo using an animal model, or clinically in humans. Such techniques are explained fully in the literature.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A heart rhythm management system, comprising:
a battery module, wherein the battery module comprises at least one connection port adapted for releasably connecting the battery module to another module;
and either or both:
a pacing module, wherein the pacing module comprises at least one connection port adapted for releasably connecting the pacing module to the battery module;
a defibrillator module, wherein the defibrillator module comprises at least one connection port adapted for releasably connecting the defibrillator module to the battery module.

2. The system of claim 1, comprising a battery module and a pacing module.

3. The system of claim 1, comprising a battery module and a defibrillator module.

4. The system of claim 1, comprising a battery module, a pacing module, and a defibrillator module.

5. The system of claim 1, wherein the battery module further comprises a capacitor module.

6. The system of claim 1, further comprising a capacitor module, wherein the capacitor module comprises at least a first and a second connection port, wherein the first connection port is adapted for releasably connecting the capacitor module to a battery module, and wherein the second connection port is adapted for releasably connecting the capacitor module to the pacing module and/or the defibrillator module.

7. The system of claim 1, wherein the modules are connected to one another with connectors

8. The system of claim 7, wherein the connectors are electrical wires or fiber optics.

9. The system of claim 7, wherein the connectors comprise electrodes for sensing, pacing, cardioversion and/or defibrillation.

10. The system of claim 7, wherein the connectors are connected to the connection port by a snap-fit connection, a clamp connection, a male-female connection, or a surface connection.

11. The system of claim 1, wherein the pacing module lacks an internal battery component.

12. The system of claim 1, wherein the defibrillator module lacks an internal battery component.

13. A method of managing a patient's heart rhythm, comprising:
implanting a battery module and a pacing module in the patient; and
releasably connecting the battery module and the pacing module, wherein the patient's heart rhythm is managed by the implanted modules.

14. The method of claim 13, wherein the battery module is remotely positioned from the pacing module.

15. The method of claim 13, wherein the battery module is implanted in the patient's abdomen.

16. The method of claim 13, wherein the modules are activated after implantation.

17. A method of managing an patient's heart rhythm, comprising:
implanting a battery module and a defibrillator module in the patient; and
releasably connecting the battery module and the defibrillator module, wherein the patient's heart rhythm is managed by the implanted modules.

18. The method of claim 17, wherein the battery module is remotely positioned from the defibrillator module.

19. The method of claim 17, wherein the battery module is implanted in the patient's abdomen.

20. A method of managing a patient's heart rhythm, comprising:
implanting a battery module, a pacing module, and a defibrillator module in the patient; and
releasably connecting the battery module, the pacing module, and the defibrillator module, wherein the patient's heart rhythm is managed by the implanted modules.

21. A method of managing a patient's heart rhythm, comprising:
implanting a defibrillator module in the patient; and
releasably connecting the defibrillator module to an existing system, wherein the existing system comprises a pacing module and a battery module, wherein the patient's heart rhythm is managed by the implanted modules.

22. A method of managing a patient's heart rhythm, comprising:
replacing a battery module in a patient's existing heart rhythm management system with a replacement battery module; and
releasably connecting the replacement battery module to the existing system, wherein the patient's heart rhythm is managed by the implanted modules.

23. An article of manufacture, comprising:
a battery module, wherein the battery module comprises at least one connection port adapted for releasably connecting the battery module to another module; and
connectors, wherein the connectors are electrical wires or fiber optics;
and either or both:
a pacing module, wherein the pacing module comprises at least one connection port adapted for releasably connecting the pacing module to a battery module; and
a defibrillator module, wherein the defibrillator module comprises at least one connection port adapted for releasably connecting the defibrillator module to a battery module.

24. The article of manufacture of claim 23, further comprising:
a capacitor module, wherein the capacitor module comprises at least a first and a second connection port, wherein the first connection port is adapted for releasably connecting the capacitor module to a battery module, wherein the second connection port is adapted for releasably connecting the capacitor module to a pacing module or a defibrillator module.

25. The system of claim 23, wherein the connectors comprise electrodes for sensing, pacing, and/or delivering high energy shocks.

26. An article of manufacture, comprising:
a battery module, wherein the battery module comprises at least one connection port adapted for releasably connecting the battery module to another module; and
connectors, wherein the connectors are electrical wires or fiber optics.

27. The system of claim 26, wherein the connectors comprise electrodes for sensing, pacing, cardioversion, and/or defibrillation.

28. An article of manufacture, comprising:
a pacing module, wherein the pacing module comprises at least one connection port adapted for releasably connecting the pacing module to a battery module; and
connectors, wherein the connectors are electrical wires or fiber optics.

29. The article of manufacture of claim 28, wherein the pacing module lacks an internal battery component.

30. The system of claim 28, wherein the connectors comprise electrodes for sensing, pacing, cardioversion, and/or defibrillation.

31. An article of manufacture, comprising:
a defibrillator module, wherein the defibrillator module comprises at least one connection port adapted for releasably connecting the defibrillator module to a battery module; and
connectors, wherein the connectors are electrical wires or fiber optics.

32. The article of manufacture of claim 31, wherein the defibrillator module lacks an internal battery component.

33. The system of claim 31, wherein the connectors comprise electrodes for sensing, pacing, cardioversion, and/or defibrillation.
